(19) United States
(12) Patent Application Publication
(10) Pub. No.: US 2025/0270155 A1
(43) Pub. Date: Aug. 28, 2025

(54) METHOD FOR PURIFYING OLEFIN GAS AND METHOD FOR PRODUCING POLYOLEFIN

(71) Applicant: JAPAN POLYPROPYLENE CORPORATION, Tokyo (JP)

(72) Inventors: Kenji MITSUTANI, Mie (JP); Taiki SATO, Chiba (JP)

(73) Assignee: JAPAN POLYPROPYLENE CORPORATION, Tokyo (JP)

(21) Appl. No.: 18/848,743

(22) PCT Filed: Mar. 23, 2023
(86) PCT No.: PCT/JP2023/011672

§ 371 Date: May 7, 2025

(30) Foreign Application Priority Data
Mar. 24, 2022 (JP) 2022-047881

(51) Int. Cl. C07C 7/11; C08F 10/02; C08F 10/06

(52) U.S. Cl. CPC C07C 7/11; C08F 10/02; C08F 10/06

(57) ABSTRACT

In a method for purifying an olefin gas in which an olefin gas containing an organoaluminum component is brought into contact with water in a scrubber to remove the organoaluminum component, by making a surface of a gas outlet peripheral edge portion of a gas nozzle for supplying the olefin gas to the scrubber have a water contact angle of 80° or more, clogging of a purification system, which is a factor that hinders a long-term continuous operation, can be prevented in a continuous process in which the olefin gas containing the organoaluminum component is brought into contact with water to precipitate and remove the organoaluminum component.

### TECHNICAL FIELD

The present invention relates to a process technique for continuously removing an organoaluminum component from an olefin gas containing the organoaluminum component. More specifically, the present invention relates to a purification method for removing an organoaluminum component contained in an unreacted olefin gas collected from a polymerization reactor for an olefin such as ethylene and propylene, and a method for producing a polyolefin using the purification method.

### BACKGROUND ART

Methods for polymerizing an olefin, for example, ethylene and propylene using a solid catalyst containing a transition metal component have been widely known. A slurry polymerization method for performing polymerization in an inert hydrocarbon solvent, a bulk polymerization method for performing polymerization in a liquefied monomer such as a liquefied propylene, and a gas phase polymerization method for performing polymerization in a gas phase in substantial absence of a liquid phase have been known as these methods for polymerizing an olefin. The gas phase polymerization method has been widely used because the gas phase polymerization method is advantageous in terms of energy costs and plant construction costs in addition to the improvement in polymerization activity, and ensures safety regarding the amount of dangerous substances retained.

In general, in a polyolefin polymerization plant, continuous operation is often performed from the viewpoint of economic rationality, and in many cases, a catalyst, a monomer, and an auxiliary agent such as an organoaluminum component are continuously supplied to a polymerization reactor, and on the other hand, a reactant gas or a slurry component is continuously extracted together with granules or powders which are polymerization products. The polymerization products obtained in the form of granules or powders are separated, subjected to a drying step, and sent to a granulator to form pellets, and a product is obtained. Here, a large amount of monomer gas is contained in the reactant gas or the slurry component extracted together with the polymerization product. However, in general, these gases are subjected to necessary purification, and then recycled and supplied again to the polymerization reactor for use.

The reactant gas collected from the polymerization reactor contains an organoaluminum component supplied as a cocatalyst or the like and a silicon compound supplied for controlling the polymerization reaction. These components may cause a pipeline to be clogged when these components come into contact with alcohols or water to precipitate a solid product or cause a decrease in heat transfer performance or clogging of a heat exchanger such as a reboiler due to precipitation of a solid product. In particular, as for organoaluminum, in the case where an unintended concentration is performed in a process, firing may occur when a pipe or a device is opened, which is extremely dangerous.

Therefore, it is required to efficiently remove the organoaluminum in the reactant gas.

In order to achieve such an object, for example, Patent Literature 1 discloses a method for removing an organoaluminum component by performing a contact treatment using a silicon oxide-containing compound whose water content is adjusted. However, the method disclosed in Patent Literature 1 has not always been industrially satisfactory in view that pressure loss of a gas flowing through a packed tower is high in addition to the fact that sufficient removal efficiency cannot be obtained, and from the economic view of exchange and construction of silicon oxide-containing compounds.

On the other hand, a method using a scrubber (a water scrubber or a washing tower) has also been known as an alternative treatment process. In the treatment process using the scrubber, the collected unreacted olefin gas, that is, the olefin gas containing the organoaluminum component is introduced from a tower bottom portion and flows to a top portion, and meanwhile, water is supplied from the top portion and the middle portion of the tower, and flows down through a filler for promoting the gas-liquid contact. The water that flows down is recycled by a pump, and an operation of discharging a part of circulating water is performed in order to keep an accumulation concentration of aluminum hydroxide generated in the tower constant.

However, when such a scrubber is operated over a long period of time, a solid product derived from the organoaluminum precipitates in a vicinity of an insert nozzle for the unreacted olefin gas, and the precipitated solid product gradually grows to clog a gas flow path. In such a case, it is necessary to stop the related process in order to perform an operation of removing the precipitated solid component, which has been a factor that hinders the long-term continuous operation. In order to solve such a problem, Patent Literature 2 discloses a method including a step of bringing an unreacted olefin gas into contact with water using a double pipe which allows the unreacted olefin gas and water to respectively flow through an inner cylinder pipe and an outer cylinder pipe, as a front stage for introducing the unreacted olefin gas into the scrubber.

### CITATION LIST

### Patent Literature

Patent Literature 1: JPH08-131707A
Patent Literature 2: JP2017-171790A

### SUMMARY OF INVENTION

### Technical Problem

However, even in the case where the method disclosed in Patent Literature 2 is used, it is not possible to completely prevent adhesion and clogging of the solid product derived from the organoaluminum in an unreacted olefin gas nozzle, and it is not necessarily satisfactory to achieve the long-term continuous operation.

In view of the above circumstances, an object of the present invention is to provide a purification method having an improved effect of preventing gas nozzle clogging that is a factor that hinders a long-term continuous operation, in a continuous process of precipitating and removing an organoaluminum component by bringing an olefin gas containing the organoaluminum component into contact with water. Further, another object of the present invention is to provide a method for efficiently producing a polyolefin without stopping a plant over a long period of time by recycling an olefin gas subjected to this process to a polymerization reactor.

### Solution to Problem

As a result of intensive studies to achieve the above objects, the present inventors have found that, when a vicinity where an olefin gas containing an organoaluminum component comes into contact with water, specifically, a surface of a gas outlet peripheral edge portion of a gas nozzle to which a solid product generated by a reaction between an organoaluminum component and water can adhere has a specific water contact angle, clogging of a gas nozzle due to the solid product can be suitably prevented. The present inventors have found that efficient production of a polyolefin can be performed by recycling an olefin gas subjected to the process to a polymerization reactor, and have completed the present invention.

The present invention provides a method for purifying an olefin gas. The method includes removing, by bringing an olefin gas containing an organoaluminum component into contact with water in a scrubber, the organoaluminum component. A surface of a gas outlet peripheral edge portion of a gas nozzle configured to supply the olefin gas to the scrubber has a water contact angle of 80° or more.

In the method for purifying the olefin gas according to the present invention, the surface of the gas outlet peripheral edge portion of the gas nozzle may be made of a material having a water contact angle of 80° or more, or may be subjected to coating processing.

In the method for purifying the olefin gas according to the present invention, from the viewpoint that the reaction between water and an organoaluminum component can be expected to be more efficiently completed, the gas nozzle includes a double pipe including an outer cylinder pipe and an inner cylinder pipe, a length of the inner cylinder pipe is smaller than that of the outer cylinder pipe, the olefin gas is caused to flow through the inner cylinder pipe, and water is caused to flow through the outer cylinder pipe. The method may include bringing the olefin gas and the water into contact with each other at a downstream side of a terminal of the inner cylinder pipe.

In the method for purifying the olefin gas according to the present invention, from the viewpoint of preventing clogging of the gas nozzle, a flow rate of the olefin gas flowing through the gas nozzle may be 5 m/s to 20 m/s.

In the method for purifying the olefin gas according to the present invention, the olefin gas may contain ethylene or propylene.

In the method for purifying the olefin gas according to the present invention, the organoaluminum component may be at least one selected from the group consisting of an alkyl aluminum halide, an alkyl aluminum hydride, an alkyl aluminum alkoxide, an alumoxane, a trialkyl aluminum, a composite organoaluminum compound, and a mixture thereof.

In the method for purifying the olefin gas according to the present invention, from the viewpoint of preventing precipitation and accumulation of solid contents in a supply pipe and a heat exchanger, and corrosion therein, the water may be at least one selected from the group consisting of desalinated water, pure water, boiler water, and distilled water.

The present invention provides a method for producing a polyolefin. The method includes purifying an olefin gas according to the method for purifying the olefin gas according to the present invention, and polymerizing a purified olefin gas.

The method for producing the polyolefin according to the present invention may include a step of purifying an unreacted olefin gas collected from a polymerization reactor according to the method for purifying the olefin gas according to the present invention and collecting the purified olefin gas, and a step of returning the collected purified olefin gas to the polymerization reactor again and performing polymerization.

### Advantageous Effects of Invention

According to the method for purifying an olefin gas of the present invention, the effect of preventing the clogging of the gas nozzle is improved in the process of purifying an olefin gas for polymerization, and therefore, a continuous operation over a longer period of time is enabled. The unreacted olefin gas collected from the polymerization reactor after the olefin polymerization reaction is subjected to the purification process according to the present invention to remove the organoaluminum component contained in the olefin gas as a residue of a cocatalyst or the like, and then the purified unreacted olefin gas is recycled to the polymerization reactor again, so that a polyolefin can be produced more continuously and efficiently than before.

### BRIEF DESCRIPTION OF DRAWINGS

FIG. 1A is a schematic view showing an example of a gas nozzle insertion structure in a device used in a purification method according to the present invention.

FIG. 1B is a schematic view showing an example of a gas nozzle insertion structure in a device used in the purification method according to the present invention.

FIG. 1C is a schematic view showing an example of a gas nozzle insertion structure in a device used in the purification method according to the present invention.

FIG. 1D is a schematic view showing an example of a gas nozzle insertion structure in a device used in the purification method according to the present invention.

FIG. 1E is a schematic view showing an example of a gas nozzle insertion structure in a device used in the purification method according to the present invention.

FIG. 1F is a schematic view showing an example of a gas nozzle insertion structure in a device used in the purification method according to the present invention.

FIG. 1G is a schematic view showing an example of a gas nozzle insertion structure in a device used in the purification method according to the present invention.

FIG. 2 is a schematic view showing an example of a gas nozzle in a device used in the purification method according to the present invention.

FIG. 3 is a schematic view showing an example of a double pipe gas nozzle in a device used in the purification method according to the present invention.

FIG. 4 is a schematic view showing an example of an inner cylinder pipe of the double pipe gas nozzle in the device used in the purification method according to the present invention.

FIG. 5 is a schematic view showing an example of an outer cylinder pipe of the double pipe gas nozzle in the device used in the purification method of the present invention.

FIG. 6 is a schematic view showing a configuration of a device used in the purification method according to the present invention.

### DESCRIPTION OF EMBODIMENTS

Embodiments of the present invention will be described below. The scope of the present invention is not limited to these descriptions, and other than the following examples, the present invention can be modified and implemented as appropriate without departing from the spirit of the present invention.

### I. Method for Purifying Olefin Gas

A method for purifying an olefin gas according to the present invention includes removing an organoaluminum component by bringing an olefin gas containing the organoaluminum component into contact with water in a scrubber. A surface of a gas outlet peripheral edge portion of a gas nozzle for supplying the olefin gas to the scrubber has a water contact angle of 80° or more.

Here, the organoaluminum component to be removed in the purification method according to the present invention is mainly used as a cocatalyst, scavenger, or the like in the polymerization of polyolefins, and is a compound that may be an undesirable content contained in an unreacted olefin gas collected from a polymerization reactor.

As for the “contact treatment between the olefin gas and water” in the purification method according to the present invention, a contact method or a structure of a device is not limited as long as the contact between the water and the gas is performed using at least a scrubber. The gas nozzle for supplying the olefin gas to the scrubber is not restricted in the form, and it is preferable that a gas introduction direction does not face the water flow.

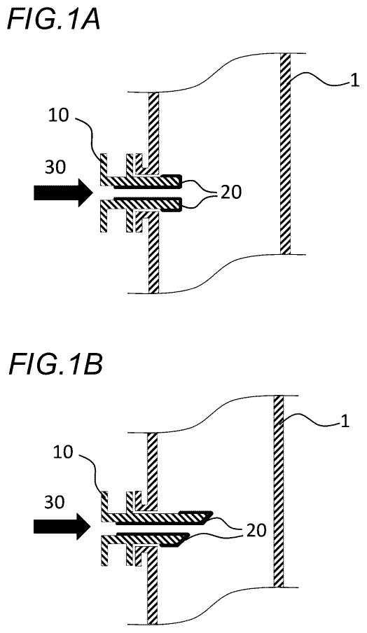

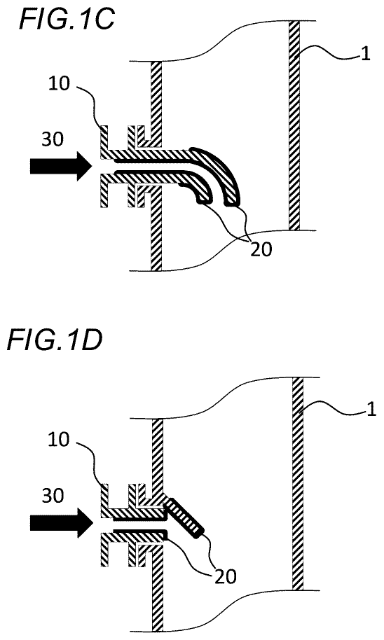

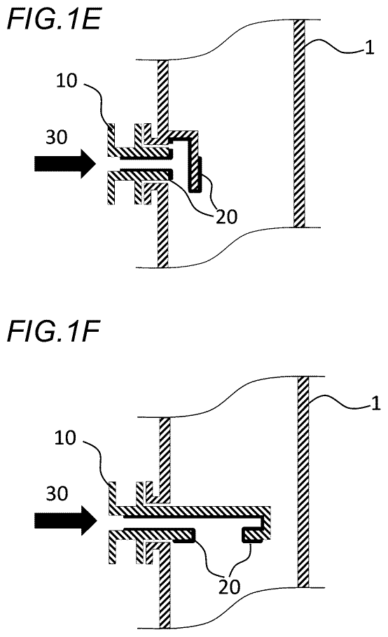

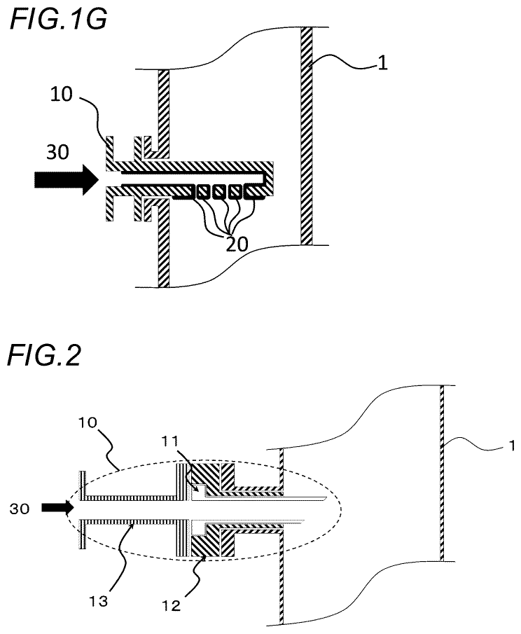

FIGS. 1A to 1G are schematic views showing examples of a gas nozzle insertion structure in a device used in the purification method according to the present invention. As the structures of the scrubber and a terminal portion of the gas nozzle inserted into the scrubber, for example, a scrubber 1 and a gas nozzle 10 having structures shown in FIGS. 1A to 1G are shown.

In the present invention, the surface of the gas outlet peripheral edge portion of the gas nozzle, in which the olefin gas containing the organoaluminum component is brought into contact with water, has a water contact angle of 80° or more.

Here, the gas outlet peripheral edge portion of the gas nozzle, which has a water contact angle of 80° or more, refers to a range in which a phenomenon that a solid product generated by a reaction between an organoaluminum component and water adheres and grows may occur.

The surface of the gas outlet peripheral edge portion of the gas nozzle, which has a water contact angle of 80° or more, is a surface to which a solid product generated by the reaction between the organoaluminum component and water may adhere.

The surface of the gas outlet peripheral edge portion of the gas nozzle, which has a water contact angle of 800 or more, can be appropriately selected on the basis of the fact that a phenomenon in which a solid product generated by the reaction between the organoaluminum component and water adheres and grows may occur on the surface.

An example of the surface of the gas outlet peripheral edge portion of the gas nozzle, which has a water contact angle of 80° or more, is a surface near a terminal portion of the gas nozzle. For example, in the gas nozzle 10 having the structures shown in FIGS. 1A to 1G, a surface 20 of a gas outlet peripheral edge portion of the gas nozzle 10 can be mentioned.

However, the surface 20 of the gas outlet peripheral edge portion of the gas nozzle 10 is not limited to the surface 20 shown in FIGS. 1A to 1G.

As shown in FIGS. 1A to 1G, examples of a portion of the surface 20 of the gas outlet peripheral edge portion of the gas nozzle, which has a water contact angle of 80° or more, include an inner surface of a terminal portion of the gas nozzle where the olefin gas containing the organoaluminum component can be first brought into contact with water, and at least a part of an outer surface of a terminal portion of the gas nozzle.

Further, it is effective to target a flow path of each of the olefin gas and water and the inner and outer surfaces of a nozzle or a pipe after the contact.

For example, a surface of a cover baffle of the scrubber at the gas outlet peripheral edge portion of the gas nozzle shown in FIGS. 1D and 1E is a surface where a phenomenon that a solid product generated by the reaction between the organoaluminum component and water adheres and grows may also occur, and therefore, its water contact angle can be set 80° or more as the surface 20 of the gas outlet peripheral edge portion of the gas nozzle.

The surface of the gas outlet peripheral edge portion of the gas nozzle, which has a water contact angle of 80° or more, may be a surface to which a solid product generated by the reaction between the organoaluminum component and water can adhere. In the case where the gas nozzle is a single pipe, examples of the surface include an inner surface at a distance of about 1 to 20 times the pipe diameter from the gas outlet of the gas nozzle, a surface of a tip portion of the gas outlet of the gas nozzle, and an outer surface at a distance of about 1 to 10 times the pipe diameter from the gas outlet of the gas nozzle.

In the present invention, a technical significance of making the surface of the gas outlet peripheral edge portion of the gas nozzle have a water contact angle of 800 or more is considered below.

The organoaluminum component in the gas reacts with water to precipitate a solid product. As a result of intensive studies, the present inventors have found that the solid product can absorb water by the capillary action because of having a porous form, and as a result, nozzle clogging is caused by repeating precipitation and growth in an upstream direction in the gas nozzle. Therefore, as a result of further adding studies for preventing the adhesion of the solid product in the nozzle or the pipe, the present inventors have known that it is effective to form the surface of the gas outlet peripheral edge portion of the gas nozzle made of a material having a water contact angle of 80° or more, or to perform coating processing with a material having a water contact angle of 80° or more, and have completed the present invention.

A gas nozzle according to the related art is made of carbon steel, stainless steel, or the like, and therefore, a surface of a terminal portion of the gas nozzle usually has a water contact angle of 70° or less, and the wettability of water is high. In contrast, in the present invention, when the surface of the terminal portion of the gas nozzle has a water contact angle of 800 or more, the surface exhibits hydrophobicity or water-repellency. It is assumed that both water and the solid product obtained by a reaction between the organoaluminum component and water hardly adhere to hydrophobic or water-repellent surfaces, and are likely to fall off. Therefore, it is considered that, with characteristics of the surface having the above water contact angle, the adhesion of the product to the gas nozzle can be suitably prevented, and the effect of preventing the gas nozzle clogging that is a factor that hinders the long-term continuous operation is improved.

The surface of the gas outlet peripheral edge portion of the gas nozzle has a water contact angle of 80° or more, and more preferably has a water contact angle of 90° or more, still more preferably has a water contact angle of 100° or more, and yet still more preferably has a water contact angle of 105° or more, from the viewpoint of improving the effect of preventing the gas nozzle clogging. On the other hand, the water contact angle of the surface of the gas outlet peripheral edge portion of the gas nozzle may be 130° or less.

The water contact angle can be measured based on a sessile drop method according to JIS R3257 (1999). For example, a measurement is performed using a contact angle meter (DMo-502) manufactured by Kyowa Interface Science Co., Ltd., 2 μL of purified water is dropped onto a smooth surface at 25° C. and 40% RH, and then after 5 seconds, a contact angle is measured.

In order to set the water contact angle to 800 or more, the surface of the gas outlet peripheral edge portion of the gas nozzle may be made of a material having a water contact angle of 80° or more, or may be subjected to coating processing with a material having a water contact angle of 80° or more.

Examples of the material having a water contact angle of 80° or more include fluorine-based resins such as polytetrafluoroethylene (PTFE), a tetrafluoroethylene-perfluoroalkyl vinyl ether copolymer (PFA), tetrafluoroethylene, and a hexafluoro propylene copolymer (FEP). In the case where the nozzle is manufactured using these materials and it is predicted that the physical strength is insufficient under the temperature and the pressure condition of the process, it is also possible to incorporate the nozzle having a terminal portion made of the material into a case including a metal nozzle or a metal pipe having a sufficient strength. For example, as shown in FIG. 2, the nozzle 11 having a terminal portion made of the material can be incorporated into a case 12 including a metal pipe or the like, and the nozzle 11, the case 12, and a gas introduction short pipe 13 can constitute the gas nozzle 10.

On the other hand, in the coating processing method in which the surface of the gas outlet peripheral edge portion has a water contact angle of 80° or more, there is no restriction as long as a predetermined water contact angle is obtained. For example, a fluorine-based coating agent for the antifouling effect is applied, and lining is performed with a fluorine-based resin such as Teflon (registered trademark) to obtain particularly good effects. Examples of the fluorine-based coating agent include, but are not limited to, SFE-DP02H, SFE-DP02HL, SNF-DP20H, and HR-FX033E manufactured by AGC Seimi Chemical Co., Ltd., A silicone-based coating agent for the antifouling effect may be used.

Any commonly known method can be appropriately selected and used as the coating or lining method.

A thickness of a coating layer formed by coating processing is not particularly limited, and may be, for example, about 1 μm to 100 μm.

In addition, the contact between the olefin gas and water may be performed in one stage. Alternatively, it is not necessary to complete the contact in one stage, and the contact between the olefin gas and water may be performed in a plurality of stages, i.e., two or more stages.

In the case where the contact between the olefin gas and water is performed in a plurality of stages, i.e., two or more stages, at least a surface of the gas outlet peripheral edge portion of the gas nozzle where the olefin gas containing the organoaluminum component in the first stage first comes into contact with water has a water contact angle of 80° or more. This is because a phenomenon in which a solid product generated by a reaction between the organoaluminum component and water adheres and grows is likely to occur.

However, in the case where the contact between the olefin gas and water is performed in a plurality of stages, i.e., two or more stages, it is preferable to make the surface of the gas outlet peripheral edge portion of the gas nozzle have a water contact angle of 80° or more when the solid product generated by a reaction between the organoaluminum component and water can adhere, not only in the first-stage contact treatment between the olefin gas and water but also in the second and subsequent contact treatments.

In the case where the contact between the olefin gas and water is performed in a plurality of stages, i.e., two or more stages, for example, a treatment of arranging two or more scrubbers in series may be performed.

Alternatively, a treatment (first stage) of bringing the olefin gas into contact with water may be performed, and then a treatment (second stage) of supplying the olefin gas to a scrubber and bringing the olefin gas into contact with water may be further performed in the scrubber. In such a case, it is possible to provide a site where the organoaluminum component in the gas reacts with water and to introduce the precipitated product derived from the organoaluminum component into the tower of the scrubber under the flow condition of the gas in the first-stage contact treatment. It is possible to complete the reaction between the organoaluminum component and water, and to collect the precipitated product to the tower bottom portion of the scrubber by spraying of water in the second-stage contact treatment.

For example, in the case where the gas nozzle includes a double pipe including an outer cylinder pipe and an inner cylinder pipe, a length of the inner cylinder pipe is smaller than that of the outer cylinder pipe, the olefin gas is allowed to flow through the inner cylinder pipe, water is allowed to flow through the outer cylinder pipe, and the olefin gas and the water are brought into contact with each other at a downstream side of a terminal of the inner cylinder pipe, the olefin gas flowing through a first flow path and the water flowing through a second flow path come into contact with each other at a downstream side of the terminal of the inner cylinder pipe, and more preferred contact is obtained. Then, it is expected that the reaction between water and the organoaluminum component can be more efficiently completed by introducing the olefin gas into the scrubber after being brought into contact with water in the pipe in advance.

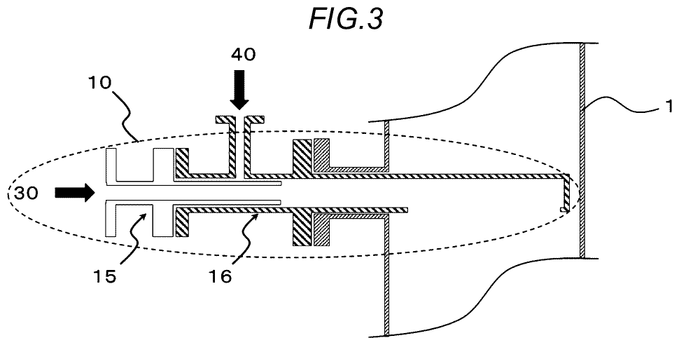

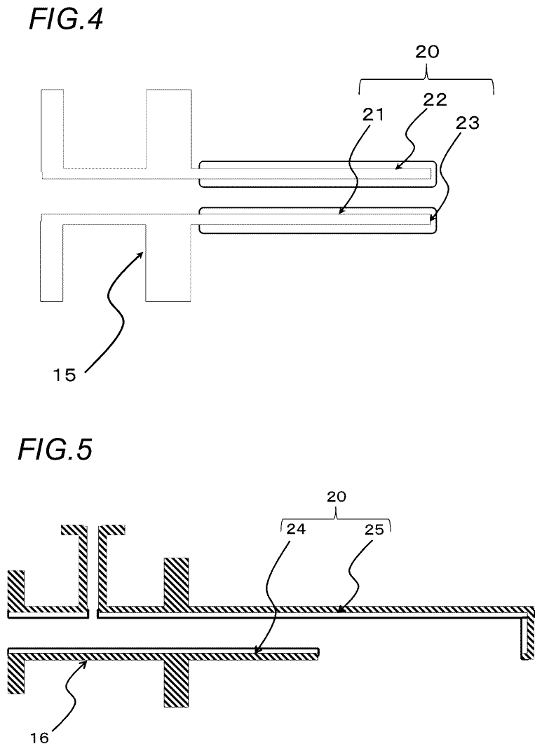

FIG. 3 is a schematic view showing an example of a gas nozzle of a double pipe in a device used in the purification method according to the present invention. In FIG. 3, the gas nozzle 10 has a double pipe including an outer cylinder pipe 16 and an inner cylinder pipe 15, a length of the inner cylinder pipe 15 is smaller than that of the outer cylinder pipe 16, the olefin gas is allowed to flow through the inner cylinder pipe 15, and the water is allowed to flow through the outer cylinder pipe 16, so that the olefin gas and the water can be brought into contact with each other at a downstream side of a terminal of the inner cylinder pipe 15. In the case where such a double pipe gas nozzle is used, as shown in FIG. 4, in the inner cylinder pipe 15, not only an inner surface 21 of the inner cylinder pipe and a tip portion 23 of the inner cylinder pipe, but also an outer surface 22 of the inner cylinder pipe correspond to a surface to which a solid product generated by a reaction between the organoaluminum component and water may adhere, and correspond to the surface of the gas outlet peripheral edge portion of the gas nozzle. Therefore, the inner surface 21, the outer surface 22, and the tip portion 23 of the inner cylinder pipe may be made of a material having a water contact angle of 80° or more, or may be subjected to the coating processing with a water contact angle of 80° or more. As shown in FIG. 5, in the outer cylinder pipe 16, inner surfaces 24 and 25 of the outer cylinder pipe correspond to a surface to which a solid product generated by a reaction between the organoaluminum component and water may adhere, and correspond to the surface of the gas outlet peripheral edge portion of the gas nozzle. Therefore, the inner surfaces 24 and 25 of the outer cylinder pipe may be made of a material having a water contact angle of 80° or more, or may be subjected to the coating processing with a water contact angle of 80° or more.

The “contact treatment between the olefin gas and water” in the purification method according to the present invention is not limited to the form of the two stages described above, and may be a multistage treatment including three or more stages. That is, it is possible to further divide the first-stage contact treatment, for example, by providing two or more double-pipe contact portions. It is also possible to further divide the second stage, for example, by providing two or more scrubbers.

The linear velocity of the olefin gas flowing through the nozzle may be designed and operated within a range of a general standard flow rate, for example, 5 m/s to 20 m/s. Under such an environment, a solid product such as aluminum hydroxide precipitated can be transferred to the scrubber without being accumulated and deposited at the terminal portion of the gas nozzle. In the scrubber, the reaction is completed by countercurrent contact between water and the gas through the filler, and the olefin gas washed by the scrubber can be discharged.

The quality of water supplied for the contact treatment is not limited as long as the water does not substantially contain a factor causing a failure in the reaction with the organoaluminum component and the washing of the gas. It is preferable to use at least one selected from the group consisting of desalinated water, pure water, boiler water, and distilled water in order to prevent precipitation and accumulation of a solid content and corrosion in a supply pipe or a heat exchanger.

As the scrubber (the water scrubber or the washing tower) used in the purification method according to the present invention, those having a known general structure in the technical field can be used. Regarding the packing in the tower, it is also possible to use Raschig rings, Lessing rings, Pall rings, Berl saddles, interlock saddles, and linear structure fillers, which are commonly used. In particular, a polypropylene structural filler which can be expected to have a large porosity and small adhesion and clogging of a solid product such as aluminum hydroxide can be suitably used. The number of fillers is preferably 15,000/m³ to 30,000/m³. A space ratio of the filler is preferably 83% to 93%. Regarding the operation conditions, when the operation can be performed by restricting the gas flow rate in the tower to the loading rate or less, a specific condition range is not determined.

The water sprayed from the tower top portion of the scrubber can be collected from the tower bottom portion and recycled, and in order to prevent the accumulation of a solid product such as aluminum hydroxide, an operation of replenishing a certain amount of water and extracting the corresponding process water may be used in combination. The amount of replenished water can be determined as appropriate based on both a concentration of an organoaluminum component contained in the olefin gas introduced into the tower and a concentration of an aluminum hydroxide component allowed in a step of treating the extracted process water.

The method for purifying an olefin gas in the present invention can be suitably used for an unreacted olefin gas collected from a reactor in a process of continuously producing a polyolefin such as polyethylene or polypropylene. As for the method for purifying an olefin gas, those devices having a known general structure in the technical field can be used. The unreacted olefin gas containing the organoaluminum component is brought into a first-stage contact with water by a double pipe configured to inject the olefin gas containing the organoaluminum component into an inner cylinder and to inject the desalinated water into an outer cylinder in a stage before supplying the unreacted olefin gas to the scrubber. The double pipe includes an outer cylinder pipe and an inner cylinder pipe and is configured to supply water between the outer cylinder pipe and the inner cylinder pipe, and the scrubber is operated by recirculating the process wastewater collected from the tower bottom portion by a circulating water pump, and performs purging so that the amount of water retained in the tower is constant. The purified olefin gas is collected from the tower top. Therefore, the olefin gas to be subjected to the purification method according to the present invention preferably contains ethylene or propylene or both ethylene and propylene.

The type of a polymerization catalyst used in the production process of the polyolefin is not particularly limited, and a known catalyst can be used. For example, a so-called Ziegler-Natta catalyst obtained by combining a titanium compound and an organoaluminum compound, or a metallocene catalyst obtained by combining a metallocene complex and an alumoxane can be used. Examples of the Ziegler-Natta catalyst include a catalyst obtained by treating and further activating, with electron donor compounds, titanium trichloride or titanium trichloride compositions obtained by reducing titanium compounds with an organoaluminum, and a so-called supported catalyst obtained by supporting titanium tetrachloride on a support such as magnesium chloride.

Examples of the organoaluminum compound used as a cocatalyst include: trialkyl aluminum such as trimethyl aluminum, triethyl aluminum, and triisobutyl aluminum; alkyl aluminum halides such as diethyl aluminum chloride, diisobutyl aluminum chloride, and ethyl aluminum sesquichloride; alkyl aluminum hydrides such as diethyl aluminum hydride; alkyl aluminum alkoxides such as diethyl aluminum ethoxide; alumoxanes such as methylalumoxanes and tetrabutylalumoxanes; and composite organoaluminum compounds such as lithium aluminum tetraethyl. One of these organoaluminum compounds may be used alone, or two or more thereof may also be mixed and used.

In addition, various polymerization additives for the purpose of improving the stereoregularity, controlling the particle property, controlling the soluble component, controlling the molecular weight distribution, and the like can be used for the above-described catalyst. Examples thereof include electro-donating compounds, for example, organic silicon compounds such as diphenyldimethoxysilane and tert-butylmethyldimethoxysilane; esters such as ethyl acetate, butyl benzoate, methyl p-toluate, and dibutyl phthalate; ketones such as acetone and methyl isobutyl ketone; ethers such as diethyl ether; organic acids such as benzoic acid and propionic acid; and alcohols such as ethanol and butanol.

Here, the organoaluminum component to be removed in the purification method according to the present invention is a compound which is used as a cocatalyst, scavenger, or the like in the polymerization of polyolefins as described above, and can be an undesirable content contained in the unreacted olefin gas collected from the polymerization reactor. Therefore, the “organoaluminum component” may be a compound listed above as an example of a cocatalyst, and is typically at least one selected from the group consisting of an alkyl aluminum halide, an alkyl aluminum hydride, an alkyl aluminum alkoxide, an alumoxane, a trialkyl aluminum, a composite organoaluminum compound, and a mixture thereof.

Ii. Method for Producing Polyolefin

The present invention also provides a method for producing a polyolefin, which is characterized in that the olefin gas is purified by the method for purifying an olefin gas according to the present invention, and further the purified olefin gas is polymerized. The method for producing a polyolefin according to the present invention preferably includes a step of purifying and collecting an unreacted olefin gas collected from a polymerization reactor, such as a bulk polymerization reactor or a gas phase polymerization reactor, by the method for purifying an olefin gas according to the present invention, and a step of performing polymerization by returning the collected purified olefin gas to the polymerization reactor again.

That is, the olefin gas from which the organoaluminum component has been removed through the method for purifying an olefin gas according to the present invention can be recycled to the polymerization reactor through a further purification process including distillation. Accordingly, a polyolefin can be continuously and efficiently produced by removing an organoaluminum component contained as a residue of a cocatalyst or the like from the unreacted olefin gas collected from the polymerization reactor, and then recycling the purified olefin gas to the polymerization reactor again.

### EXAMPLE

Hereinafter, the present invention will be described in more detail with reference to Examples, but the present invention is not limited thereto.

### EXAMPLE

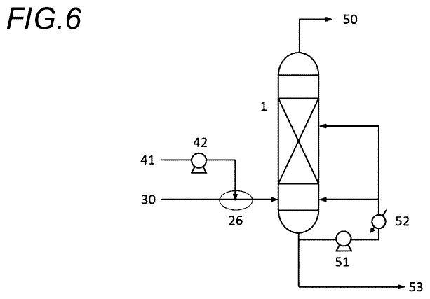

For 60 m³/h of an olefin gas collected from a reactor for continuously polymerizing propylene using triethyl aluminum as a cocatalyst, a purification treatment was carried out using a scrubber for the purpose of removing an organoaluminum component. A concentration of triethyl aluminum in the olefin gas was 140 ppm by mass. A device configuration shown in FIG. 6 was used. The scrubber had an inner diameter of 450 mm and packed with a polypropylene S-type Tellerette (registered trademark, Tsukishima Kankyo Engineering Ltd.) in a layer height of 4300 mm. As shown in FIG. 6, in a stage before supplying the olefin gas to the scrubber, the olefin gas was brought into a first-stage contact with water by a double pipe configured to inject the olefin gas containing organoaluminum into an inner cylinder and to inject desalinated water into an outer cylinder.

An example of the double pipe includes a double pipe, in which the inner cylinder pipe and the outer cylinder pipe as shown in FIG. 3 were used, a pipe having an outer diameter of 89.1 mm and an inner diameter of 78.1 mm was used as the outer cylinder pipe, and a pipe having an outer diameter of 60.5 mm and an inner diameter of 52.7 mm was used as an inner cylinder pipe. In each of the inner cylinder pipe and the outer cylinder pipe, the sites corresponding to the surface 20 of the gas outlet peripheral edge portion shown in FIGS. 4 and 5 were separately coated with a fluorine-based antifouling coating agent. The water contact angle of the surface coated with the fluorine-based antifouling coating agent was 105°. For the material of the double pipe, a carbon steel was used for both the inner cylinder pipe and the outer cylinder pipe. However, under the condition that the antifouling coating agent was not applied, the double pipe was likely to be wet with water, and the water contact angle was almost 0°. With a structure in which water is supplied between the outer cylinder pipe and the inner cylinder pipe, the operation was conducted with a water supply rate of 50 L/h. The flow rate of the olefin gas in the inner cylinder pipe was about 7.6 m/s. The scrubber was operated by circulating, by a circulating water pump, the process wastewater collected from the tower bottom portion, and performed purging so that the amount of water retained in the tower is constant. In the purged process water, a concentration of the particulate substance having a diameter of 2 mm or less was 500 mg/L or less. The propylene polymerization was performed for one year under this condition, but there was no clogging of the olefin gas insert nozzle during the operation, and the scrubber was able to operate stably. The gas supply insert nozzle to the double pipe and the scrubber was opened and inspected, but no deposition or clogging of a solid content was found.

### Comparative Example

As Comparative Example, an operation was performed under the same conditions as those of the above Example except that the fluorine-based antifouling coating agent was not applied and the water contact angle of the surface of the gas outlet peripheral edge portion of the gas nozzle was substantially 0°. As a result, the olefin gas did not flow after about three months from the start of the operation, and the operation was disabled. When the operation was stopped and the scrubber was opened, it was found that the precipitated aluminum hydroxide adhered to and clogged the inner pipe of the double pipe.

### <Consideration Based on Comparison Between Example and Comparative Example>

It was shown that the olefin gas did not flow after about three months from the start of the operation, and the operation was disabled in the comparative example, and in contrast, the gas flow path was prevented from being clogged even after one year or longer from the start of operation, and a long-term continuous operation was enabled in the example. From the comparison between the results of the example and the comparative example, the significance of the configuration of the present invention is demonstrated, and the superiority of the present invention with the related art is also clear.

The gist of the present invention is as follows.

<1> A method for purifying an olefin gas, the method including: removing an organoaluminum component by bringing an olefin gas containing the organoaluminum component into contact with water in a scrubber, in which a surface of a gas outlet peripheral edge portion of a gas nozzle configured to supply the olefin gas to the scrubber has a water contact angle of 80° or more.

<2> The method for purifying the olefin gas according to <1>, in which the surface of the gas outlet peripheral edge portion of the gas nozzle is made of a material having a water contact angle of 80° or more, or is subjected to coating processing with a material having a water contact angle of 80° or more.

<3> The method for purifying the olefin gas according to <1> or <2>, in which the gas nozzle includes a double pipe including an outer cylinder pipe and an inner cylinder pipe, a length of the inner cylinder pipe is smaller than a length of the outer cylinder pipe, the olefin gas is caused to flow through the inner cylinder pipe, and water is caused to flow through the outer cylinder pipe, the method including bringing the olefin gas and the water into contact with each other at a downstream side of a terminal of the inner cylinder pipe.

<4> The method for purifying the olefin gas according to any one of <1> to <3>, in which a flow rate of the olefin gas flowing through the gas nozzle is 5 m/s to 20 m/s.

<5> The method for purifying the olefin gas according to any one of <1> to <4>, in which the olefin gas contains ethylene or propylene.

<6> The method for purifying the olefin gas according to any one of <1> to <5>, in which the organoaluminum component is at least one selected from the group consisting of an alkyl aluminum halide, an alkyl aluminum hydride, an alkyl aluminum alkoxide, an alumoxane, a trialkyl aluminum, a composite organoaluminum compound, and a mixture thereof.

<7> The method for purifying the olefin gas according to any one of <1> to <6>, in which the water is at least one selected from the group consisting of desalinated water, pure water, boiler water, and distilled water.

<8> A method for producing a polyolefin, the method including: purifying an olefin gas according to the method for purifying the olefin gas according to any one of <1> to <7>; and polymerizing a purified olefin gas.

<9> The method for producing the polyolefin according to <8>, further including: purifying an unreacted olefin gas collected from a polymerization reactor according to the method for purifying the olefin gas according to any one of <1> to <7> and collecting the purified olefin gas; and returning the collected purified olefin gas to the polymerization reactor again and performing polymerization.

The present application is based on a Japanese Patent Application (No. 2022-047881) filed on Mar. 24, 2022, the contents of which are incorporated into this application by reference.

### INDUSTRIAL APPLICABILITY

In the method for purifying an olefin according to the present invention, by making the surface of the gas outlet peripheral edge portion of the gas nozzle for supplying the olefin gas to the scrubber have a water contact angle of 800 or more, clogging of the insert nozzle of the scrubber can be prevented, and the long-term continuous operation can be performed, so that the purification method is beneficial in the polyolefin production industry.

In the method for producing a polyolefin according to the present invention, the unreacted olefin gas collected from the polymerization reactor after the olefin polymerization reaction is subjected to the purification process according to the present invention to remove the organoaluminum component contained in the olefin gas as a residue of a cocatalyst or the like, and then the purified unreacted olefin gas is recycled to the polymerization reactor again, so that a polyolefin can be produced more continuously and efficiently than before, which is beneficial.

### REFERENCE SIGNS LIST

1 scrubber
10 gas nozzle
11 nozzle having terminal portion made of material having water contact angle of 80° or more
12 case
13 gas introduction short pipe
15 inner cylinder pipe
16 outer cylinder pipe
20 surface of gas outlet peripheral edge portion
21 inner surface of inner cylinder pipe
22 outer surface of inner cylinder pipe
23 tip portion of inner cylinder pipe
24 inner surface of outer cylinder pipe
25 inner surface of outer cylinder pipe
26 double pipe
30 unreacted olefin gas containing an organoaluminum component
40 water
41 desalinated water
42 desalinated water pump
50 purified olefin gas not containing organoaluminum component
51 circulating water pump
52 circulating water heat exchanger
53 process wastewater

1. A method for purifying an olefin gas, the method comprising:
removing an organoaluminum component by bringing an olefin gas containing the organoaluminum component into contact with water in a scrubber,
wherein a surface of a gas outlet peripheral edge portion of a gas nozzle configured to supply the olefin gas to the scrubber has a water contact angle of 800 or more.

2. The method for purifying the olefin gas according to claim 1,
wherein the surface of the gas outlet peripheral edge portion of the gas nozzle is made of a material having a water contact angle of 800 or more, or is subjected to coating processing with the material having a water contact angle of 800 or more.

3. The method for purifying the olefin gas according to claim 1 or 2,
wherein the gas nozzle includes a double pipe including an outer cylinder pipe and an inner cylinder pipe, a length of the inner cylinder pipe is smaller than a length of the outer cylinder pipe, the olefin gas is caused to flow through the inner cylinder pipe, and water is caused to flow through the outer cylinder pipe, the method comprising:
bringing the olefin gas and the water into contact with each other at a downstream side of a terminal of the inner cylinder pipe.

4. The method for purifying the olefin gas according to any one of claims 1 to 3,
wherein a flow rate of the olefin gas flowing through the gas nozzle is 5 m/s to 20 m/s.

5. The method for purifying the olefin gas according to any one of claims 1 to 4,
wherein the olefin gas contains ethylene or propylene.

6. The method for purifying the olefin gas according to any one of claims 1 to 5,
wherein the organoaluminum component is at least one selected from the group consisting of an alkyl aluminum halide, an alkyl aluminum hydride, an alkyl aluminum alkoxide, an alumoxane, a trialkyl aluminum, a composite organoaluminum compound, and a mixture thereof.

7. The method for purifying the olefin gas according to any one of claims 1 to 6,
wherein the water is at least one selected from the group consisting of desalinated water, pure water, boiler water, and distilled water.

8. A method for producing a polyolefin, the method comprising:
purifying an olefin gas according to the method for purifying the olefin gas according to any one of claims 1 to 7; and
polymerizing a purified olefin gas.

9. The method for producing the polyolefin according to claim 8, further comprising:
purifying an unreacted olefin gas collected from a polymerization reactor according to the method for purifying the olefin gas according to any one of claims 1 to 7 and collecting the purified olefin gas; and
returning the collected purified olefin gas to the polymerization reactor again and performing polymerization.
